(19) United States
(12) Patent Application Publication
(10) Pub. No.: US 2015/0306269 A1
(43) Pub. Date: Oct. 29, 2015

(54) UPSTREAM HYDROGEN PEROXIDE NEBULIZING SANITATION SYSTEM

(71) Applicant: The Vincit Company, LLC, Chattanooga, TN (US)

(72) Inventors: Robert C. Bullard, Signal Mountain, TN (US); Battle Glascock, Soddy Daisy, TN (US); James A. Faller, Chattanooga, TN (US)

(21) Appl. No.: 14/627,711

(22) Filed: Feb. 20, 2015

Related U.S. Application Data
(60) Provisional application No. 61/985,852, filed on Apr. 29, 2014.

(51) Int. Cl. A61L 9/14; A61L 9/20

(52) U.S. Cl. CPC A61L 9/14; A61L 9/20

(57) ABSTRACT

Sanitation systems for treating an airstream, such as disinfection, sterilization, and/or deodorization, by ionization are disclosed. In embodiments, the air sanitation system is disclosed having an inlet and an outlet with a plenum between them. An ultraviolet (UV) light source is arranged within the plenum and provides UV light to an area of the plenum. A hydrated quad-metallic catalyst having a desiccant is disposed in the area, and a nebulizer is arranged between the inlet and the UV light source. The nebulizer can provide a nebulized agent within the plenum, and the UV light can generate reactive oxygen species, such as hydroxyl radicals, from water captured by the desiccant or from the nebulized agent.

### RELATED APPLICATION

The present application claims the benefit of U.S. Provisional Application No. 61/985,852, filed Apr. 29, 2014, which is incorporated herein in its entirety by reference.

### TECHNICAL FIELD

In embodiments, the invention relates to methods and apparatuses for disinfection, sterilization, and/or deodorization of air, by ionization. This can be accomplished by use of chemical apparatuses and/or processes for disinfecting, deodorizing, preserving, and/or sterilizing treated airstreams. In particular, the present invention is directed to a sanitation system that can provide continual treatment, particularly a sanitation system with a chemical intervention solution comprising, for example, hydrogen peroxide, nebulized upstream from one or more photohydroionization cells. Such treated airstreams can be used to treat hard surfaces and/or food products that are being processed, such as protein foods (i.e., poultry, red meat, eggs, fruits and vegetables).

### BACKGROUND

It is well known that bacteria and fungi are found in air and are transported from one surface to another by air currents. Bacteria and fungi can also be found in food processing plants, such as on hard surface sources, as well as on the food product that is being processed. While many different applications have been used to obtain sanitation, there still is a need for systems that are more efficient, effective, reliable and robust. In particular, there is the need for a system that can reduce the quantity or kill undesirable microorganisms in the air before they settle onto food products and food contact surfaces.

### SUMMARY

In some aspects, the present invention is directed at a sanitation system that can provide continual treatment to food products, such as red meat, poultry, eggs, fruit and/or vegetables, and/or food contact surfaces. In some aspects, the sanitation system continually treats a source of air, while in some other aspects, the sanitation system can be used to treat a source of food product being processed. In some aspects, the sanitation system of the present invention utilizes hydrogen peroxide that has been nebulized upstream from one or more photohydroionization cells. In some aspects, the sanitation system of the present invention utilizes a chemical solution that has been nebulized upstream from one or more photohydroionization cells, particularly where the chemical solution comprises a peroxycarboxylic acid, such as peroxyacetic acid.

In some aspects, a sanitation system includes a housing defining a plenum having an inlet and an outlet, an ultraviolet (UV) light source arranged within the plenum and configured to provide UV light to an area of the plenum between the inlet and the outlet, and a desiccant disposed in the proximate area of the UV light source. A nebulizer is arranged between the inlet and the UV light source and configured to provide a nebulized agent within the plenum, wherein the UV light has an energy level high enough to generate reactive oxygen species, such as hydroxyl radicals, from water captured by the desiccant or from the nebulized agent.

According to another embodiment, a method of treating a flow of an airstream comprises collecting an airstream at an inlet of a plenum having the inlet and an outlet, providing a nebulized agent to the airstream by a nebulizer arranged at least partially in the plenum, and routing the airstream to a desiccant subsystem. At the desiccant subsystem, UV radiation can be applied to the desiccant. The UV radiation can have a sufficiently low wavelength to cause water captured by the desiccant to ionize into reactive oxygen species, such as hydroxyl radicals. The resultant airstream, including the generated reactive oxygen species (i.e., hydroxyl radicals), can then be emitted at the outlet.

According to yet another embodiment, a method of sanitizing food products, such as poultry, red meat, hatchery eggs, fruit or vegetables, using a sanitation system includes providing a sanitation system in a facility containing the food products. A housing defines a plenum having an inlet and an outlet. A UV light source is arranged within the plenum and configured to provide UV light to an area of the plenum between the inlet and the outlet. A nebulizer is arranged between the inlet and the UV light source, and a desiccant is disposed in the area. An airstream is collected at the inlet, and a nebulized agent is provided to the airstream by the nebulizer. The airstream is routed to the desiccant, the UV light is applied to the desiccant to generate the reactive oxygen species, such as hydroxyl radicals, from water contained within the airstream and/or nebulized agent captured by the desiccant, and the airstream is emitted, including the generated reactive oxygen species at the outlet, whereby the treated airstream is applied to the food products.

In some aspects, the nebulized agent nebulized agent comprises hydrogen peroxide, a peroxycarboxylic acid, or mixtures thereof. In some aspects, the peroxycarboxylic acid is an equilibrium peroxycarboxylic acid solution. In some other aspects, the peroxycarboxylic acid is a pH modified peroxycarboxylic acid solution. In some aspects, the peroxycarboxylic acid is chosen fom peroxyformic acid, peroxypropionic acid, peroxyacetic acid, peroxybutanoic acid, peroxypentanoic acid, peroxyhexanoic acid, peroxyheptanoic acid, peroxyoctanoic acid, peroxynonanoic acid, peroxydecanoic acid, peroxyundecanoic acid, peroxydodecanoic acid, peroxylactic acid, peroxymaleic acid, peroxyascorbic acid, peroxyhydroxyacetic acid, peroxyoxalic acid, peroxymalonic acid, peroxysuccinic acid, peroxyglutaric acid, peroxyadipic acid, peroxypimeli acid, peroxysubric acid, and mixtures thereof. In some other aspects, the peroxycarboxylic acid comprises peroxyacetic acid.

### BRIEF DESCRIPTION OF THE DRAWINGS

FIG. 1 is a perspective view of a sanitation system according to certain embodiments of the present invention.

FIG. 2 a perspective view of certain internal components of the sanitation system of FIG. 1 according to certain embodiments of the present invention.

FIGS. 3A-3C are side and end view of a sanitation system according to certain embodiments of the present invention.

FIG. 4 is a perspective view of an illustrative photohydroionization cell used in a sanitation system according to certain embodiments of the present invention.

FIG. 5 is an end view of the illustrative photohydroionization cell in FIG. 4 with the photohydroionization service access cover removed according to certain embodiments of the present invention.

FIG. 6 is a perspective view of a sanitation system according to an embodiment incorporating a nebulizer.

FIG. 7 is a cross-sectional view of a flow blurring nebulizer according to certain aspects of the present invention.

FIGS. 8A-8D depict the sanitation system of FIG. 6 in various plan views with cutaways to show internal components.

FIG. 9 is a flowchart of a method for sanitizing an airstream, according to an embodiment of the present invention.

### DETAILED DESCRIPTION OF THE DRAWINGS

The sanitation system of the present invention can be used as an air sanitation system that provides continual treatment of plant air through the utilization of a specially designed air handler and a process referred to as Photohydroionization (PHI). In some aspects, the sanitation system of the present invention addresses airborne sources of microbes (i.e., bacteria and/or fungi), which are typically overlooked by processors in lieu of hard surface sources. In some other aspects, the sanitation system of the present invention addresses bacteria and/or fungi on processing food products, such as poultry (i.e., chicken, turkey, fowl), red meat (i.e., beef, pork), fruits, vegetables and other sources of protein food products (i.e., eggs, nuts). In some aspects, the sanitation system of the present invention is used to provide an airstream having antimicrobial activity on hatchery eggs. In any of these applications, the sanitation system of the present invention provides a very reliable and robust alternative to more costly and problematic ozone generators or chemical spray systems.

The sanitation system of the present invention uses the UV/H2O2 Advanced Oxidation Process (AOP). AOP is a process in which reactive oxygen species, such as free radical hydroxyls (.OH) and ozone are generated, and upon their recombination in the presence of a catalyst hydro peroxides are generated through the decomposition of H₂O. The process requires the use of a specialized desiccant material which includes the catalyst material. In some embodiments, the catalyst material may comprise titanium dioxide. In some aspects, the catalyst material is applied to reflectors proximate a UV radiation source. In certain aspects, the desiccant is positioned in close proximity to a UV radiation source emitting energy at a specific wavelengths set to maximize the process effect.

Ambient air where the air sanitation system is located is drawn through the air sanitation system, where the water in the ambient air is absorbed by the desiccant material. Once absorbed, the water from the air is brought into contact with the catalyst and exposed to the UV radiation. When this moisture is exposed to UV radiation, and allowed ample time to absorb the UV radiation while retained on the desiccant material comprising the catalyst, several processes can be initiated. In some aspects, the desiccant material may comprise the catalyst.

While in the presence of ultraviolet (UV) light, the water is broken into hydroxyl radicals (OH). The oxidation potential of a hydroxyl radical (2.8V) is much greater than ozone (2.07V) and chlorine (1.39V) and thus has the capability of oxidizing a variety of organic and inorganic contaminants, including the cellular structures of bacteria and fungi. The recombination of the hydroxyl and ozone free radicals in the presence of the catalyst, promotes the development of hydrogen peroxide. UV light catalyzes the dissociation of hydrogen peroxide into hydroxyl radicals through chain reactions or the hydrogen peroxide is entrained into the air stream where it destroys bacteria and fungi. This in combination with the dislodged free radicals and the UV radiation means the sanitation system utilizes four different bacteria and/or fungi killing agents in a very safe and effective way.

The combination of safe low level ozone (O₃), hydroxyls, and a broad spectrum UV light enhanced by a hydrated quad-metallic compound that comprises the desiccant and catalyst produces an advanced oxidation reaction. This process also produces other reactive species, such as hydro-peroxides, super oxide ions, ozonide ions and hydroxides. By providing the proper UV light wavelength, the PHI Cell provides safe hydro-peroxides, super oxide ions, ozonide ions and hydroxides to purify the airstream.

In some aspects, instead of sanitizing air flowing through this system, the treated airstream from this system, including the four killing agents created by the system, can be applied through the airstream contacting food products such as red meat, poultry, fruits, vegetables, eggs, hatching eggs, or other protein food products, to kill surface microorganisms contained on such food products. Depending on the food product, food products may remain stationary in the airstream or be conveyed through the airstream on suitable conveyor mechanisms.

In some aspects, the sanitation system of the present invention utilizes a nebulizing system that “seeds” the incoming airstream with a chemical intervention solution prior to the PHI cell for a dramatically increase volume of free radical generation. Such a nebulizing system can be used in air sanitation and/or the food processing applications discussed above. By providing ultra-small droplets of the chemical intervention solution, such as 0.5-10% hydrogen peroxide (H₂O₂) in the incoming airstream, there are more available reactive oxygen species, such as free radical hydroxyls, to perform the desired work of sanitizing the airstream and/or the food product source. In some aspects, the specialized nebulizing injector provides about 0.5 to about 5.0 micron droplets, in some aspects about 1.0 to about 2.5 micron droplets, in some other aspects about 1.5 to about 2.0 micron droplets, and in some other aspects about 1.7 micron droplets of the chemical intervention solution, such as a peroxygen compound, on a continuous basis with the supply delivered to all units through on chemical delivery system positioned in a convenient location. One of ordinary skill in the art will appreciate that the nebulizing injector can provide different sized droplets of the chemical intervention solution depending upon the specific application and the composition of the chemical intervention solution.

In some aspects, the specialized nebulizing injector provides about 0.5 to about 5.0 micron droplets of hydrogen peroxide, in some aspects about 1.0 to about 2.5 micron droplets of hydrogen peroxide, in some other aspects about 1.5 to about 2.0 micron droplets of hydrogen peroxide, and in some other aspects about 1.7 micron droplets of hydrogen peroxide, on a continuous basis with the supply delivered to all units through on chemical delivery system positioned in a convenient location. One of ordinary skill in the art will appreciate that the nebulizing injector can provide different sized droplets of the chemical intervention solution comprising hydrogen peroxide depending upon the specific application.

In some aspects, the chemical intervention solution consumption rate is approximately 1.9 Liters per minute with effective flows as low as 2 mL/hr, depending on organic loading of the food products. In some aspects, the chemical intervention solution consumption rate is about 0.5 Liters per minute to about 3.0 Liters per minute. In some aspects, the effective flow is between about 2 mL/hour and about 5 mL/hour. One of ordinary skill in the art will appreciate that the consumption rate is dependent upon unit size, volume of air treated, and operating time per day, such that values outside these ranges are contemplated.

In some aspects, the hydrogen peroxide consumption rate is approximately 1.9 Liters per minute with effective flows as low as 2 mL/hour, depending on organic loading of the food products. In some aspects, the hydrogen peroxide consumption rate is about 0.5 Liters per minute to about 3.0 Liters per minute. In some aspects, the effective flow is between about 2 mL/hour and about 5 mL/hour. In some aspects, the nebulized hydrogen peroxide requires the supply of about a 0.5-10% solution of hydrogen peroxide, or more preferably about 3-8% solution of hydrogen peroxide. One of ordinary skill in the art will appreciate that the consumption rate is dependent upon unit size, volume of air treated, and operating time per day, such that values outside these ranges are contemplated.

The process of using the sanitization system of the present invention also provides continual circulation of plant air in large volumes. When properly designed for a given space, and with consideration for air distribution based on unit placement within the space, the destruction of microbiology and odors is the positive result.

With the sanitation system of the present invention, micro-organisms (i.e., bacteria and/or fungi) can be reduced up to about 60%, in certain aspects up to about 70%, in certain aspects up to about 80%, in certain aspects up to about 90%, in certain aspects up to about 95%, in certain other aspects up to about 99%, and in certain other aspects up to about 99.99% in certain situations. Gases, Volatile Organic Compounds (VOCs), and odors can also be reduced significantly, and the air within the plant will contain ozonide ions, hydro-peroxides, super oxide ions and hydroxides, which will provide continuous protection for the air as well as equipment without the use of relatively more temperamental open electrode plasma or corona discharge ozone generation systems. In certain aspects, the sanitation system of the present invention utilizes air handles that are easily mounted within the plant space and suspended from the ceiling with mounting lugs.

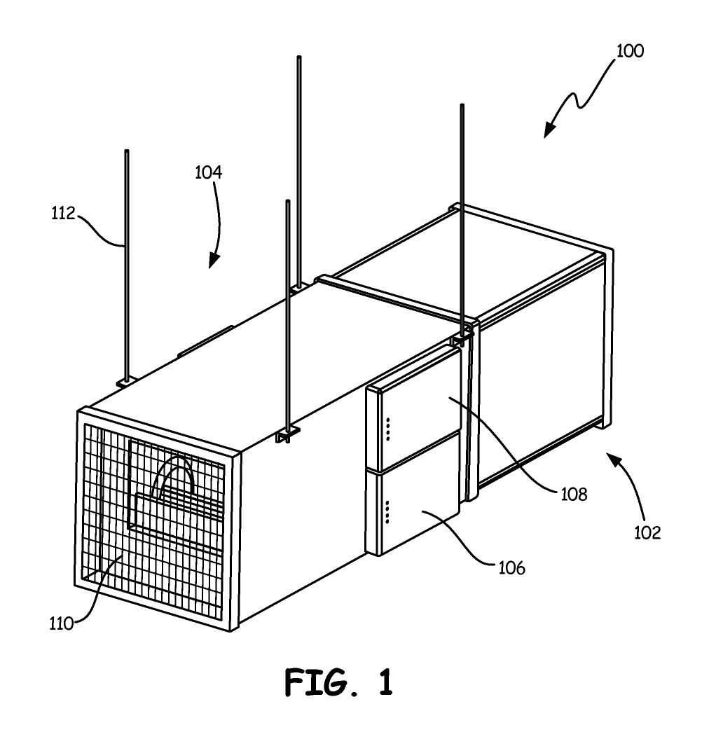

Referring now to the Figures, FIG. 1 is a perspective view of sanitation system 100, according to an embodiment of the invention. Sanitation system 100 is a system for the treatment of air, such as air within a food product processing plant. Sanitation system 100 generates an airstream, which is cleaned and/or provides a source of “scrubbers” such as hydroxyl radicals or other reactive oxygen species.

Sanitation system 100 includes inlet portion 102 and outlet portion 104. Outlet portion 104 further comprises first PHI tube section 106 and second PHI section 108, and grating 110. In the embodiment shown in FIG. 1, sanitation system 100 is configured to hang from some other structure, such as a ceiling, by supports 112. In other aspects, sanitation system 100 may be configured to rest on a bench top or other support structure instead of being hung from a ceiling.

Inlet portion 102 is a portion of the sanitation system 100 through which air can be collected that is routed through the sanitation system 100 in an airstream. In general, inlet portion 102 provides ingress for air to be treated by sanitation system 100. In some embodiments, inlet portion 102 can comprise gratings or filters, such as particulate filters, carbon filters, or electret filters, that provide some level of treatment to an airflow that passes through sanitation system 100.

Outlet portion 104 is the portion of sanitation system 100 arranged downstream of inlet portion 102. Outlet portion 104, in combination with inlet portion 102, provides the radially outer part of a plenum through which an airstream can pass during operation of sanitation system 100. Several other structures can be provided, interior to outlet portion 104, which facilitate the operation of sanitation system 100.

One such interior structure, in embodiments, is one or more PHI tubes. In the embodiment shown in FIG. 1, sanitation system 100 includes first PHI tube section 106 and second PHI tube section 108, although in alternative embodiments more or fewer PHI tube sections can be used. In the embodiment shown in FIG. 1, the PHI tube sections 106 and 108 are accessible from the exterior of the sanitation system 100, and extend through the plenum defined by inlet portion 102 and outlet portion 104. PHI tube sections 106 and 108 each include at least one UV light source and a desiccant (not shown in this Figure).

Grating 110 is positioned near the far downstream end of sanitation system 100. Grating system 110 can prevent objects from traveling into the plenum from the downstream end of outlet portion 104. In alternative embodiments, grating 110 can be a filter or a wire grating, for example.

In operation, an airstream travels into inlet portion 102, and then into outlet portion 104, passing through at least one of PHI tube sections 106 and 108 along the way before leaving sanitation system 100 through grating 110. Contaminants in the airstream, such as bacteria or fungi, are treated as they pass through sanitation system 100. Furthermore, the airstream leaving sanitation system 100 contains reactive oxygen species, such as hydroxyl radicals and/or other sanitizing agents. This can be accomplished by several mechanisms.

First, in embodiments having filter media at inlet portion 102, particulates can be filtered out of the airstream. Second, first PHI tube section 106 and second PHI tube section 108 in conjunction with the catalyst convert water or other molecules present in the airstream into hydroxyl radicals and/or other “scrubber” radicals that sanitize the air and any other materials that the airstream comes into contact with, including bacteria and/or fungi in the air. Third, the PHI tube sections 106 and 108 emit ultraviolet light which can kill bacteria or fungi passing through the PHI tube sections 106 and 108.

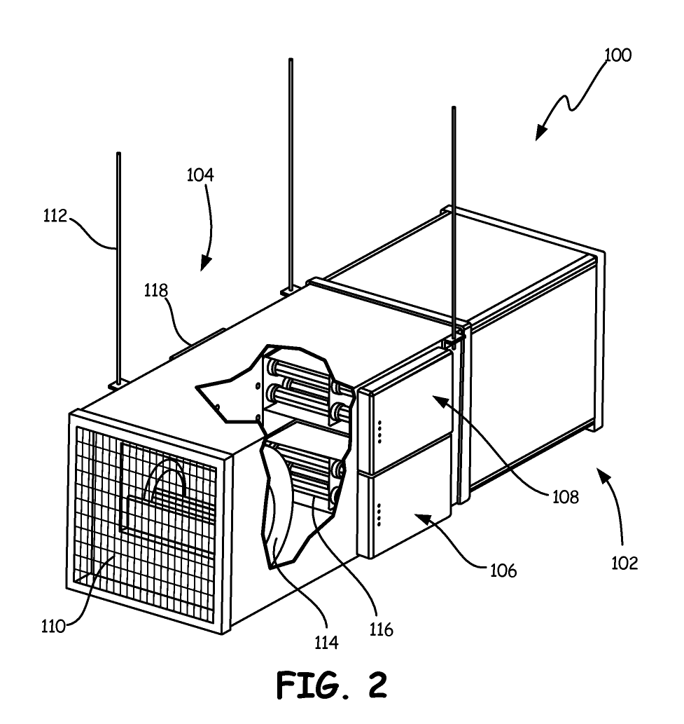

FIG. 2 shows the sanitation system 100 of FIG. 1, with some portions of the outlet portion 104 cut away to show certain components that are internal to the plenum defined by inlet portion 102 and outlet portion 104. In particular, the internal components include blower 114, PHI tubes 116, and power supply 118.

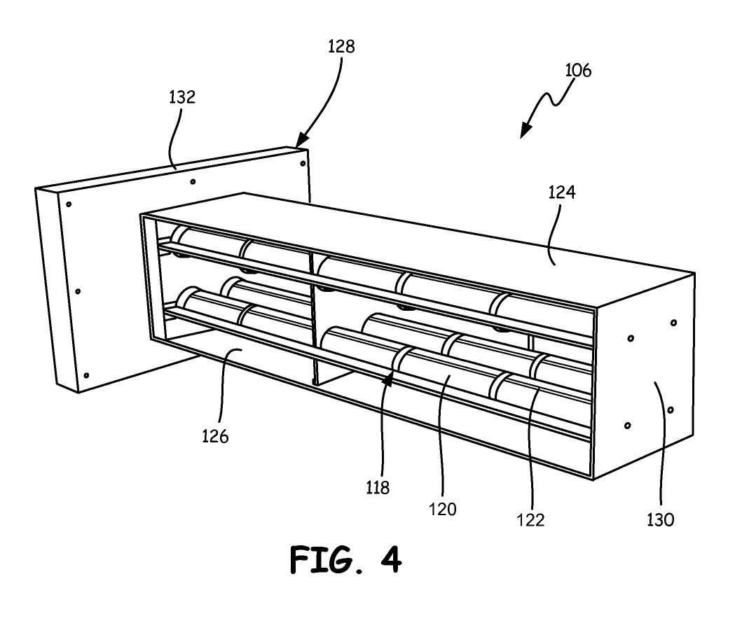

Blower 114 draws an airstream through sanitation system 100, the airstream being drawn into inlet portion 102 and to outlet portion 104 and out through grating 110. An airstream that passes through sanitation system 100 in this way travels past a plurality of PHI tubes 116, in the embodiment shown in FIG. 1. Each PHI tube 116 comprises a UV light source with a hydrated quad-metallic compound, which comprises a desiccant material and a catalyst, as shown in more detail with respect to FIG. 4. As such, an airstream passing through sanitation system 100 will be filtered at inlet portion 102, and be UV light treated at PHI tube sections 106 and 108. Water in the airstream can be absorbed by the desiccant material in proximity to each of PHI tube sections 106 and 108, and utilizes the catalyst with UV light to dissociate into reactive oxygen species, such as hydroxyl radicals, before continuing downstream to blower 114 and out through grating 110.

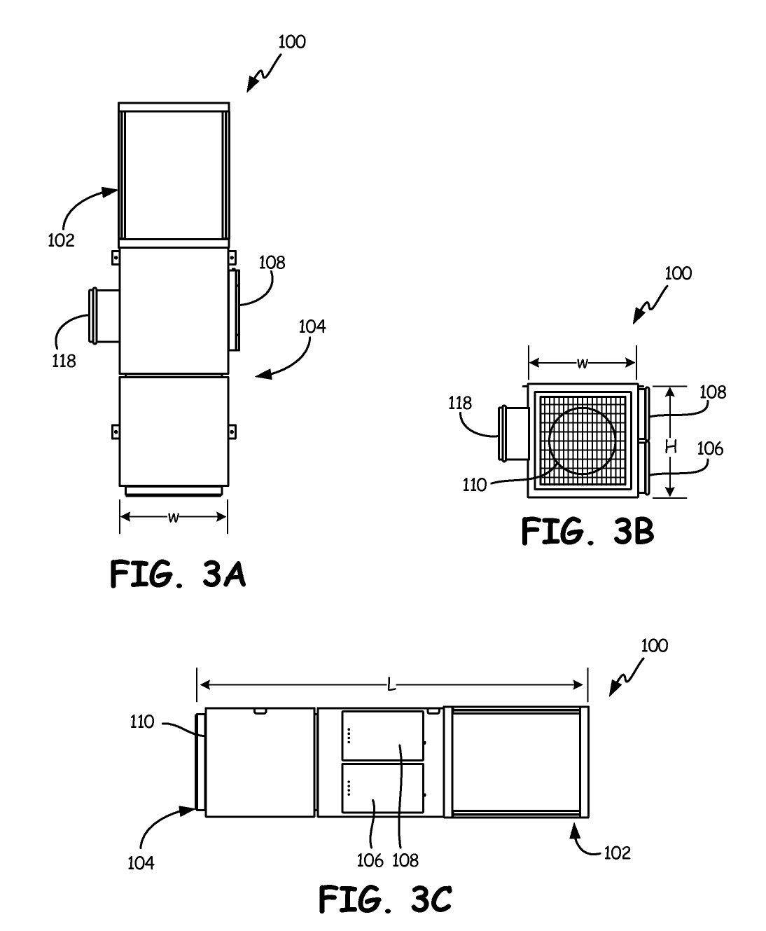

FIGS. 3A-3C depict various plan views of sanitation system 100 from top, downstream, and side directions, respectively. FIGS. 3A-3C include many of the features previously described with respect to FIGS. 1 and 2, such as inlet portion 102, outlet portion 104, PHI tube sections 106 and 108, grating 110, and power supply 118. Additionally, as shown in FIGS. 3A-3C, sanitation system 100 has a length L, a width W, and a height H. The dimensions of sanitation system 100, in various embodiments, can be selected to provide suitable airflow and sanitation for any given air sanitation process or desired room space. In one embodiment, for example, length L can be about 237.5 cm (93.5 in.), width W can be about 66 cm (26 in.), and height H can be about 67.67 cm (26.64 in.). One of ordinary skill in the art will appreciate that the foregoing dimensions are not intended to be limiting, but provide an illustration of an embodiment.

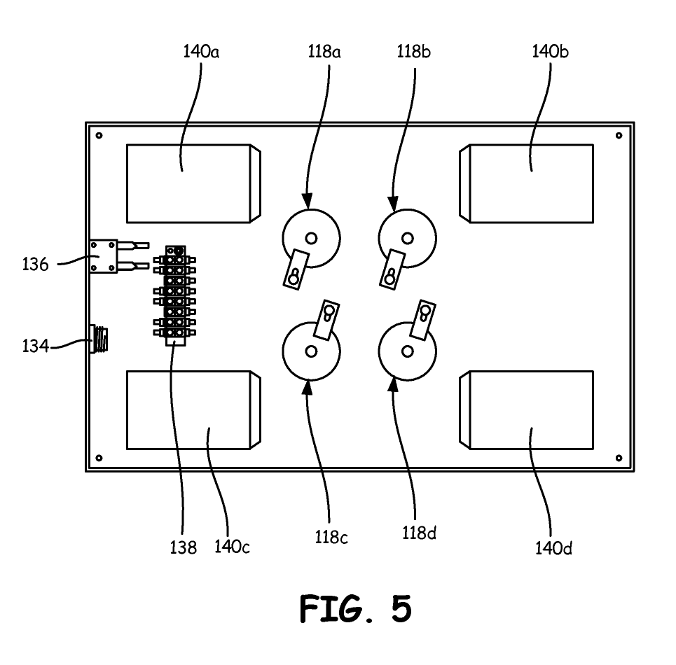

FIG. 4 is a perspective view of first PHI section 106, according to an embodiment. First PHI section 106 comprises PHI tubes 118, each having a UV light source 120 and longitudinal strips 122. UV light sources 120 and longitudinal strips 122 extend between PHI cell guards 124 and 126 in a first direction, and between plug 128 and end cap 130 in another direction. Plug 128 can interface with the exterior wall of the plenum through which the airstream flows, as shown for example in FIGS. 1 and 2 wherein plug 128 of first PHI tube section 106 is exterior to outlet portion 104 while the remainder of first PHI tube 106 is housed within the plenum defined by inlet portion 102 and outlet portion 104. Plug 128 further comprises electronic enclosure 132, which is described in more detail with respect to FIG. 5.

With continued reference to FIG. 4, PHI tubes 118 extend through the plenum defined by inlet portion 102 and outlet portion 104 and sanitize an airstream passing through it. UV light sources 120 provide some level of disinfection of the air stream, by killing airborne bacteria and fungi. Furthermore, in some embodiments, longitudinal strips 122 can contain a material such as a desiccant that captures water in the airstream and the catalyst, such as titanium dioxide. Water held in longitudinal strips 122 can be dissociated into two hydroxyl radicals that are released into the airstream and continue to sanitize the airstream and objects incident to it. In other embodiments, longitudinal strips can contain a hydrated quad-metallic compound comprising a desiccant material and a catalyst, which the catalyst in the presence of UV light creates reactive oxygen species, such as hydroxyl radicals. As such, the PHI tubes 118 can produce hydro peroxides, super oxide ions, hydroxides, passive negative ions and ozonide ions from water in the airstream passing through.

FIG. 5 is a cutaway view of the interior of the plug 128 of FIG. 4, in particular showing the components housed within electronic enclosure 132 of FIG. 4. As shown in FIG. 5, the electrical cables connecting various components are not depicted, for clarity. Those of skill in the art will understand that the various components described herein could be coupled to one another using a variety of electrical cords and/or cables to provide power to the four PHI tubes 118a, 118b, 118c, and 118d.

FIG. 5 illustrates fitting 134 that allows for an electrical cable (not shown) to be routed to circuit breaker 136. From circuit breaker 136, power can be delivered to bus 138. Bus 138 routes appropriate levels of power to the four ballast boxes 140a, 140b, 140c, and 140d. Each of the four ballast boxes 140a-140d are electrically coupled to one of the PHI tubes 118a-118d. In this way, the UV lights described with reference to FIGS. 1-4 are powered.

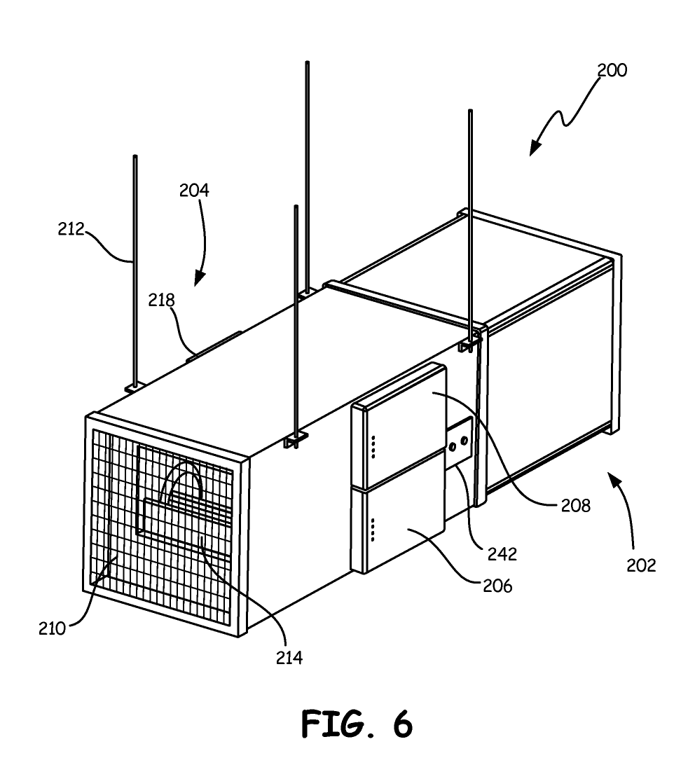

FIG. 6 is a perspective view of an alternative embodiment, sanitation system 200. Sanitation system 200, like sanitation system 100 previously described with reference to FIGS. 1-5, includes an inlet portion 202, outlet portion 204, first PHI tube section 206, second PHI tube section 208, grating 210, supports 212, fan 214, PHI tubes 216 (not shown in this view), and power supply 218. These components function in a substantially similar fashion to their counterparts in sanitation system 100. Additionally, sanitation system 200 includes a means for dissociating the chemical intervention solution into a fine mist, vapor, spray or aerosol, such as a nebulizer, atomizer, micronizer or fogger, which allows the delivery of the chemical intervention solution having particles sizes between about 4 microns and about 50 microns.

As shown in FIG. 6, the means for vaporizing or providing the chemical intervention solution in the form of a mist is nebulizer 242, which provides a nebulized agent upstream of first PHI tube section 206 and second PHI tube section 208. Nebulizer 242 can provide the nebulized agent in a mist, vapor, spray or aerosol form. For example, nebulizer 242 can provide a substance that dissociates, either alone or in combination with water, into various “scrubber” agents such as hydroxyl radicals.

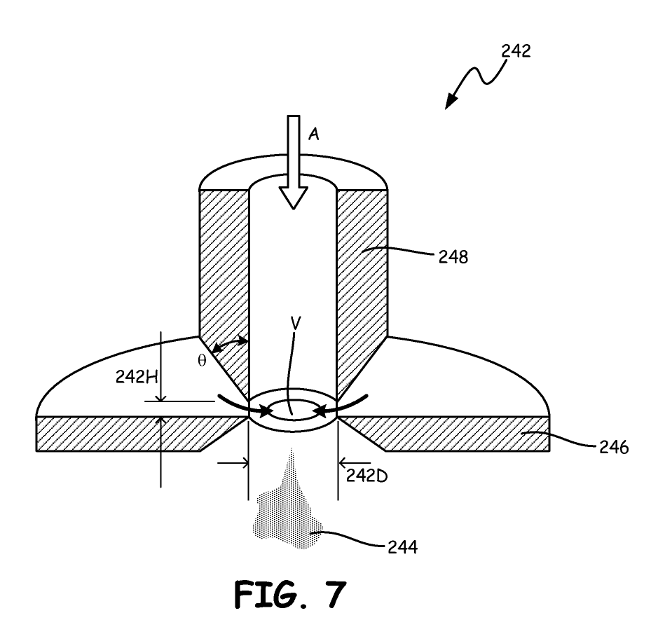

FIG. 7 is a cross-sectional view of nebulizer 242 in operation. In particular, FIG. 7 depicts nebulizer 242 providing nebulized agent 244 in an airstream A. The nebulized agent 244 is provided at an outlet of the nebulizer 242 adjacent to a flange portion 246. Flange portion 246 and inlet tube 248 are coupled at a vortex region V. Airflow A passes through inlet tube 248, mixes in vortex region V, and then leaves nebulizer 242 on the far side of flange portion 246. As depicted in FIG. 7, inlet tube 248 extends at an angle θ from vortex region V, and has a uniform internal diameter 242D. In addition to the angle θ, vortex region V provides mixing and voracity based on height 242H of the vortex region V.

Nebulizer 242 can be used to introduce a wide variety of nebulized agents 244, at a wide variety of flow rates. For example, nebulized agent 244 can be hydrogen peroxide in some embodiments, introduced at a rate of about 2 mL/hr. In some other aspects, the nebulized agent 244 can comprise a chemical intervention solution. In some aspects, the chemical intervention solution comprises hydrogen peroxide.

In some aspects, the chemical intervention solution that makes up the nebulized agent 244 comprises an equilibrium peroxycarboxylic acid. In some aspects, the equilibrium peroxycarboxylic acid has a pH above about 3.0 and below about 7.0, in certain aspects a pH range of about 3.5 to about 5.5, and in some other aspects a pH range of about 3.5 to about 5.0. In certain preferred aspects of the present invention, the equilibrium peroxycarboxylic acid comprises peroxyacetic acid.

Peroxyacetic acid, sometimes also called peracetic acid or PAA, is a peroxycarboxylic acid having a strong oxidizing potential, has the molecular formula CH₃COOOH, and has a molecular structure as follows:

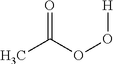

An equilibrium peroxyacetic acid solution is produced from an equilibrium mixture of hydrogen peroxide, acetic acid and water (“equilibrium PAA solution”), which often uses an acid catalyst, e.g., sulfuric acid.

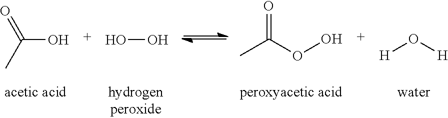

In some other aspects, the chemical intervention solution that makes up nebulized agent 244 comprises a pH modified peroxycarboxylic acid. In some aspects, the pH modified peroxycarboxylic acid has a pH above about 7.0 and below about 10.0, in certain aspects a pH range of about 7.0 to about 9.5, and in some other aspects a pH range of about 7.5 to about 9.0. In certain preferred aspects of the present invention, the pH modified peroxycarboxylic acid comprises peroxyacetic acid. In certain aspects of the present invention, the pH modified peroxycarboxylic acid is prepared using at least one buffering agent, said at least one buffering agent chosen from sodium hydroxide, potassium hydroxide, sodium salts of carbonic acid, potassium salts of carbonic acid, phosphoric acid, silicic acid and combinations thereof. In such preparation, the buffering agent is added to an equilibrium peroxycarboxylic acid solution until the pH is between above about 7.0 and below about 10.0.

In some aspects, the peroxycarboxylic acid for the equilibrium peroxycarboxylic acid solution and/or the pH modified peroxycarboxylic acid solution is chosen from peroxyformic acid, peroxypropionic acid, peroxyacetic acid, peroxybutanoic acid, peroxypentanoic acid, peroxyhexanoic acid, peroxyheptanoic acid, peroxyoctanoic acid, peroxynonanoic acid, peroxydecanoic acid, peroxyundecanoic acid, peroxydodecanoic acid, peroxylactic acid, peroxymaleic acid, peroxyascorbic acid, peroxyhydroxyacetic acid, peroxyoxalic acid, peroxymalonic acid, peroxysuccinic acid, peroxyglutaric acid, peroxyadipic acid, peroxypimeli acid, peroxysubric acid, and mixtures thereof.

In some other aspects, the concentration of the chemical intervention solution is preferably between about 10 ppm and about 5000 ppm, in some aspects between about 25 ppm and about 2500 ppm, in some aspects between about 50 ppm and about 1500 ppm, in some aspects between about 75 ppm and about 1000 ppm, and in some other aspects between about 100 ppm and about 750 ppm.

In some aspects, the nebulized agent 244 comprises a mixture of hydrogen peroxide and a peroxycarboxylic acid having 1-18 carbon atoms. In some aspects, the nebulized agent 244 comprises a mixture of hydrogen peroxide and peroxyacetic acid.

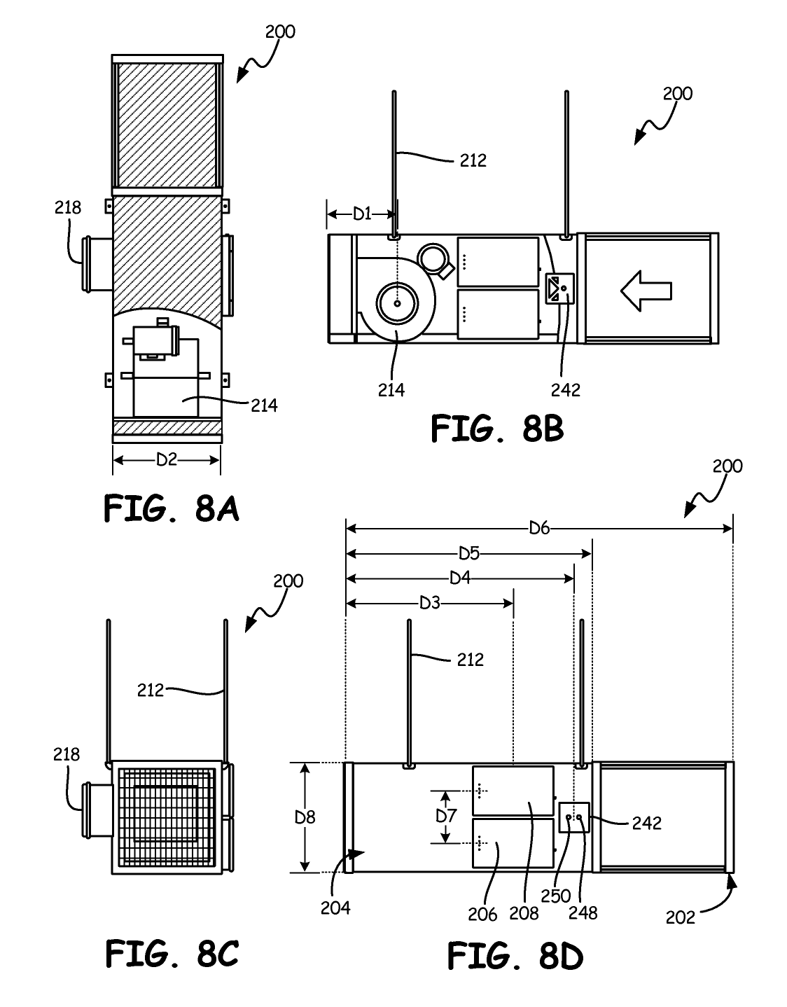

FIGS. 8A-8D are various plan views of sanitation system 200, showing dimensions of sanitation system 200, according to one embodiment. Furthermore, FIGS. 8A and 8B are partially cut away to show internal features of sanitation system 200.

FIG. 8A is a top view of sanitation system 200, with a partial cutaway showing the position of blower 214 relative to the rest of the sanitation system 200. In particular, sanitation system 200 has a width dimension D2 that is based on the size of sanitation system 200 (excluding power supply 218). Blower 214 is approximately centered in sanitation system 200 with respect to width dimension D2, as shown.

FIG. 8B is a side view of sanitation system 200, which can be suspended by supports 212 in a similar fashion to the supports 112 previously described. In particular, FIG. 8B is a cutaway view, depicting fan 214 and nebulizer 242. An arrow shows the expected direction of flow of an airstream during operation of sanitation system 200, driven by fan 214. FIG. 8B also depicts dimension D1, the distance between the output end of sanitation system 200 and the center of rotation of blower 214.

FIG. 8C is an end view of sanitation system 200, depicting power supply 218 extending from one side.

FIG. 8D is a side view of sanitation system 200, without cutaway, depicting various dimensions. In particular, FIG. 8D depicts dimension D3, which is the distance from the outlet end of sanitation system 200 and the center of first PHI tube section 206 and second PHI tube section 208. FIG. 8D further depicts dimension D4, which is the distance from the outlet end of sanitation system 200 and the center of nebulizer 242. FIG. 8D further depicts dimension D5, which is the distance across outlet section 204. FIG. 8D further depicts dimension D6, which is the total length of sanitation system 200. FIG. 8D further depicts dimension D7, which is the distance between the center of first PHI tube section 206 and the center of second PHI tube section 208 (dimension D7 is perpendicular to the direction of the other dimensions, D3-D6, previously described with respect to this Figure). Dimension D8 is the total height of sanitation system 200.

In one embodiment, the approximate dimensions D1-D8 of sanitation system 200 are as follows:

### D1=41.42 cm (16.31 in.)

### D2=66 cm (26 in.)

### D3=103 cm (40.55 in.)

### D4=140.23 cm (55.21 in.)

### D5=151.25 cm (59.55 in.)

### D6=238.5 cm (93.9 in.)

### D7=32.08 cm (12.63 in.)

### D8=66.04 cm (26 in.)

In alternative embodiments, of course, the dimensions could be smaller or larger, depending on the level of airflow needed to properly sanitize an environment or a product passing through an outlet airstream. In addition to varying the absolute sizes of these dimensions, the relative proportions of the dimensions can be modified for various purposes, such as to fit in a particular space or to generate a desired size, speed, and/or voracity of airflow.

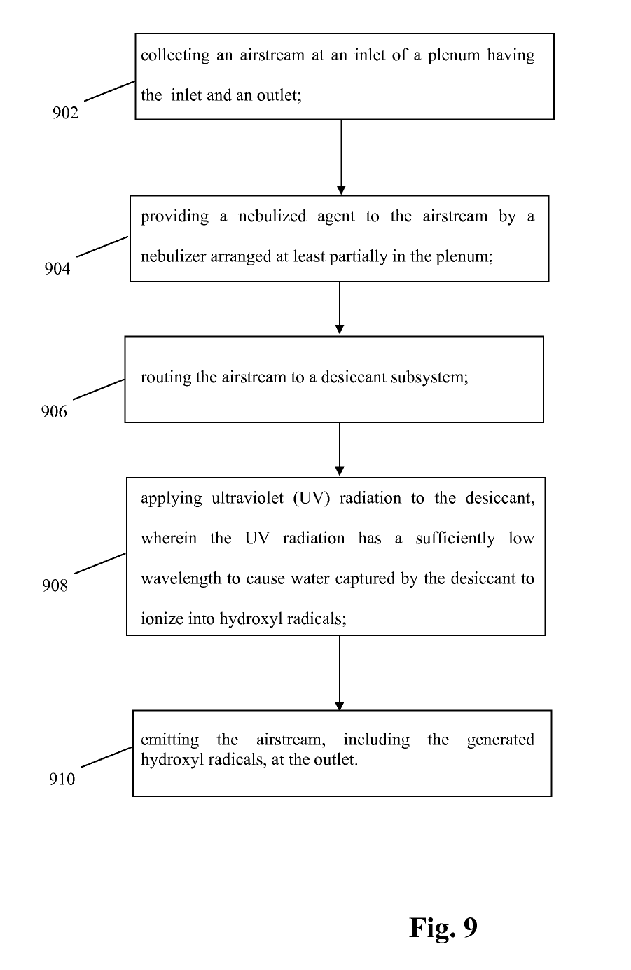

FIG. 9 is a flowchart of a method for using a sanitation system, such as sanitation system 200 previously described.

At step 902, an airstream is collected at an inlet of a plenum having an inlet and an outlet. As shown in the previously-described figures, this could include collecting air at inlet portion 202, for example.

At step 904, a nebulized agent is provided to the airstream by a nebulizer arranged at least partially in the plenum. For example, as shown in the previously-described figures, nebulizer 242 is arranged partially inside the plenum defined by inlet portion 202 and outlet portion 204. The nebulized agent, described in more detail with respect to FIG. 7, is provided within the airstream.

At step 906, the airstream is routed to a desiccant subsystem. The desiccant subsystem could be, for example, a PHI tube system, such as first PHI tube system 206 and second PHI tube system 208. As used throughout this application, it should be understood that desiccant can include a catalyzing agent, including a catalyzing agent of a hydrated quad-metallic compound.

At step 908, UV light is applied to the desiccant. The UV light applied has a sufficiently low wavelength to cause water (or, in embodiments, the nebulized agent) to ionize into hydroxyl radicals.

At step 910, the airstream, including any hydroxyl radicals generated by the UV light and the desiccant system, is emitted from the outlet (e.g., outlet portion 204 of the previously-described figures).

### EXAMPLES

According to preliminary test results, the systems and methods previously described are effective at reducing the quantity of undesirable contaminants, such as bacteria and/or fungi.

### Example 1

### Egg Shell Testing

In preliminary testing, twelve store-bought fresh eggs were prepared by blowing out the contents and cutting the egg shells in half. Twenty-four half shells were inoculated by submersion in a broth solution containing about a seven log concentration bacteria. Egg shells were removed and allowed to air dry. The shells were separated into groups of six, one group designated to be exposed to air running through the air sanitizing system containing only nebulized 3% hydrogen peroxide, another group exposed to UV light-generated radicals, and the last group exposed to both 3% hydrogen peroxide and UV radicals. The final six shells were designated the control group and were exposed only to untreated air circulated though the sanitation system. The twenty-four half shells were allowed to be exposed to their respective situation for a period of 10 minutes.

Immediately following treatment, approximately 1 square inch swab samples were taken from the surface of each egg shell then serially diluted and plated on Tryptic Soy Agar and incubated for 24 hours to obtain a total plate count. Results indicate that treatment with H₂O₂ alone results in a 1.5 log₁₀ reduction, UV radical treatment alone resulted in a 2.14 log₁₀ reduction, and the combination of both hydrogen peroxide and UV radical treatment resulted in a 2.53 log₁₀ reduction.

### Example 2

### Chicken Wings

Similar testing was performed on chicken wings. Twenty-four store-bought chicken wings were prepared by submersing them in a Tryptic Soy Broth broth solution containing about a seven log concentration of bacteria. The wings were removed and allowed to air dry. The wings were separated into groups of six, one group designated to be exposed to air running through the air sanitation system containing only nebulized 3% hydrogen peroxide, another group exposed to UV light-generated radicals, and the last group exposed to both 3% H₂O₂ and UV radicals. The final six wings were designated the control group and were exposed only to untreated air circulated though the sanitation system.

Immediately following treatment at times of 10 seconds, 30 seconds, 45 seconds and 60 seconds, approximately 1 square inch of skin on each wing was swabbed with moistened sample sponges. The sponges were stomached in phosphate buffer solution for 2 minutes, then the buffer solution was serially diluted and plated on Tryptic Soy Agar and incubated for 24 hours to obtain a total plate count.

Results indicate that treatment with H₂O₂ alone results in a maximum 0.9 log₁₀ reduction, UV radical treatment alone resulted in a maximum 1.42 log₁₀ reduction, and the combination of both hydrogen peroxide and UV radical treatment resulted in a maximum 1.75 log₁₀ reduction.

Accordingly, the treatment of samples with both hydrogen peroxide seeding prior to the UV light sources resulted in significant antimicrobial activity compared to hydrogen peroxide treatment alone or UV light-generated radical treatment alone.

1. A sanitation system comprising:
a housing defining a plenum having an inlet and an outlet;
an ultraviolet (UV) light source arranged within the plenum and configured to provide UV light to an area of the plenum between the inlet and the outlet;
a coating comprising a desiccant material and a catalyzing agent disposed proximate the UV light source; and
a vaporizing means for transforming a chemical intervention solution into an aerosol, the vaporizing means arranged between the inlet and the UV light source and configured to provide the aerosol within the plenum,
wherein the UV light has an energy level high enough to generate a reactive oxygen species from water captured by the desiccant or from the aerosol.

2. The sanitation system of claim 1, and further comprising a filter arranged at the inlet.

3. The sanitation system of claim 1, wherein the chemical intervention solution comprises hydrogen peroxide.

4. The sanitation system of claim 1, wherein the chemical intervention solution comprises a peroxycarboxylic acid.

5. The sanitation system of claim 4, wherein the chemical intervention solution comprises 10-500 parts per million of the peroxycarboxylic acid.

6. The sanitation system of claim 1, wherein the chemical intervention solution comprises a mixture of hydrogen peroxide and a peroxycarboxylic acid.

7. The sanitation system of claim 3, wherein the vaporizing means is a nebulizer.

8. The sanitation system of claim 7, wherein the nebulizer provides the hydrogen peroxide in the form of the aerosol having a particle size between about 1.5 microns and about 50 microns.

9. The sanitation system of claim 1, wherein the vaporizing means is chosen from a nebulizer, an atomizer, a micronizer, and a fogger.

10. The sanitation system of claim 9, wherein the vaporizing means is a flow blurring nebulizer.

11. The sanitation system of claim 1, wherein the sanitation system comprises a plurality of PHI cells.

12. A method of treating a flow of an airstream, the method comprising:
collecting an airstream at an inlet of a plenum having the inlet and an outlet;
providing an chemical intervention solution in the form of a aerosol to the airstream at least partially in the plenum;
routing the airstream to a desiccant subsystem;
applying ultraviolet (UV) radiation to the desiccant, wherein the UV radiation has a sufficiently low wavelength to cause water from the airstream captured by the desiccant or the aerosol captured by the desiccant to react with a catalyst to form one or more reactive oxygen species; and
emitting the airstream, including the generated one or more reactive oxygen species, at the outlet.

13. The method of claim 12, wherein the chemical intervention solution in the form of the aerosol is hydrogen peroxide.

14. The method of claim 12, wherein the chemical intervention solution in the form of the aerosol comprises peroxycarboxylic acid.

15. The method of claim 14, wherein the chemical intervention solution in the form of the aerosol comprises peroxycarboxylic acid at a concentration between about 10 ppm and about 500 ppm.

16. The method of claim 12, wherein the vaporizing means is chosen from a nebulizer, an atomizer, a micronizer, and a fogger.

17. A method of treating a food product, the method comprising:
providing an air sanitation system in a facility containing food products, the air sanitation system comprising:
a housing defining a plenum having an inlet and an outlet;
an ultraviolet (UV) light source arranged within the plenum and configured to provide UV light to an area of the plenum between the inlet and the outlet;
a nebulizer arranged between the inlet and the UV light source; and
a hydrated quad-metallic compound comprising a desiccant material and a catalyzing agent, disposed in the area;
collecting an airstream at the inlet;
providing a nebulized agent in the form of an aerosol, mist, vapor or fog to the airstream by the nebulizer;
routing the airstream to the hydrated quad-metallic compound;
applying the UV light to the hydrated quad-metallic compound to generate one or more reactive oxygen species from water captured by the desiccant or the chemical intervention solution; and
emitting the airstream, including the generated one or more reactive oxygen species, at the outlet.

18. The method of claim 17, wherein the nebulized agent comprises hydrogen peroxide.

19. The method of claim 17, wherein the nebulized agent comprises a peroxycarboxylic acid.

20. The method of claim 17, wherein the food product is chosen from poultry, red meat, fruits, vegetables, eggs, hatchery eggs and nuts.
